(19) United States
(12) Patent Application Publication
(10) Pub. No.: US 2017/0281650 A1
(43) Pub. Date: Oct. 5, 2017

(54) USE OF DIHYDROCHOLESTEROL

(71) Applicants: Lei Guan, Beijing (CN); Youyou Zhao, Beijing (CN); Junkuan Wang, Beijing (CN); NESTEC S.A., Vevey (CH)

(72) Inventors: Lei Guan, Beijing (CN); Youyou Zhao, Beijing (CN); Junkuan Wang, Beijing (CN)

(21) Appl. No.: 15/508,278

(22) PCT Filed: Sep. 2, 2014
(86) PCT No.: PCT/CN2014/085746

§ 371 Date: Mar. 2, 2017

(51) Int. Cl. A61K 31/575; A61K 9/00; C12Q 1/60

(52) U.S. Cl. CPC A61K 31/575; C12Q 1/60; A61K 9/0053

(57) ABSTRACT

The use of dihydrocholesterol (DHC) in low doses of up to 50 mg per kg of body weight to treat or prevention excess weight or obesity and/or hyperlipidemia, and/or a disorders associated with any of the foregoing, and a composition comprising DHC.

### FIELD OF THE INVENTION

The invention relates to the use of a low dose of dihydrocholesterol to treat and/or prevent excess weight or obesity, and/or hyperlipidemia, and/or a disorder associated with any of the foregoing. The invention further relates to compositions comprising DHC.

### BACKGROUND OF THE INVENTION

Hyperlipidemia refers to a condition wherein the plasma levels of one or more lipid in a subject's plasma, e.g. a mammal's plasma lipid level, is abnormally elevated above the norm. Hyperlipidemia may for example refer to hypercholesterolemia, hypertriglyceridemia, or any combination thereof.

A variety of disorders are associated with hyperlipidemia. For example an elevated plasma cholesterol level in a subject is amongst the main risk factors for Cardiovascular Diseases (CVDs). According to a World Health Organization (WHO) report, an estimated 17.3 million people died from CVDs in 2008, representing 30% of all global deaths and making them the leading causes of death and disability globally.

Excess weight and obesity are also risk factors for CVDs, as well as for a variety of other disorders, e.g. metabolic disorders such as Type II diabetes.

Excess weight and obesity refer to conditions wherein a subject e.g. a mammal has an excess of body fat. Body Mass Index (hereinafter BMI) is a measure frequently used to assess whether a subject has excess weight or is obese. In humans a person is classed as being overweight or having excess weight if they have a BMI from 25 to 30. If a human has a BMI over 30 they are classed as being obese.

For most subjects suffering from excess weight or obesity, and/or hyperlipidemia e.g. hypercholesterolemia, hypertriglyceridemia and combinations thereof, dietary recommendations and exercise are the first line of therapy, but these measures alone are often not sufficiently effective and often some form of medication is necessary. However, known medicines, particularly in the doses needed to be effective, can suffer from drawbacks i.e. unwanted side effects.

Accordingly, there is a need for medicines that can be used to treat and/or prevent excess weight or obesity, and/or hyperlipidemia that do not suffer from all of the drawbacks of the prior art.

Surprisingly, it has now been found that dihydrocholesterol (hereinafter “DHC”) may be used in a low dose to minimise weight gain and/or to lower plasma lipid levels, in particular plasma triglyceride levels, plasma cholesterol levels and a combination thereof. This finding stems from an investigation of the effect of a low dose of DHC on Golden Syrian hamsters. This hamster model is known to be particularly suited for studying lipid metabolism, in particular the effect on plasma cholesterol levels of functional foods (see Zesheng Zhang et al., 2009; Choosing hamsters but not rats as a model for studying plasma cholesterol-lowering activity of functional foods; Molecular Nutrition & Food Research Volume 53, Issue 7, pages 921-930). To the inventors' knowledge said study was the first of its kind to identify the hypolipidemic effects of DHC at a low dose where side effects are nonexistent or the risk thereof is minimised.

The effects of DHC on plasma cholesterol levels has previously been studied using several other animal models e.g. cockerels, rats and rabbits. However, these studies employed higher doses of DHC than those disclosed herein. Said high doses have an increased risk of side effects e.g. bile stones and abnormalities in platelet function, associated with them. Further, the acceptability of these animal models for studying cholesterol metabolism is questionable.

Accordingly, there was no indication that DHC could be used in a low dose, which minimises and/or avoids the risk of side effects, as disclosed herein.

### SUMMARY OF THE INVENTION

The invention is set out in the claims. The inventors have found that a low dose of DHC can be used to minimise weight gain and/or to lower plasma lipid levels, in particular plasma cholesterol levels, and/or plasma triglyceride levels. Accordingly DHC in a low dose can be used to treat and/or prevent excess weight or obesity and/or hyperlipidemia, in particular hypercholesterolemia and/or hypertriglyceridemia, and disorders associated with any of the foregoing for example; lipoprotein dysregulation, hyperlipidemia related cardio-cerebro-vascular diseases including coronary heart disease, angina, myocardial infarction, atherosclerosis, coronary artery disease, stroke, claudication, peripheral vascular disease, non-alcohol fatty liver disease, metabolic diseases such as type II diabetes and combinations thereof.

The low dose of DHC may be used to minimise weight gain and/or to lower plasma lipid levels, in particular plasma cholesterol levels, and/or plasma triglyceride levels, in a subject e.g. a human, a cat or a dog.

The low dose of DHC may be up to 50 mg per kg of bodyweight, in particular the low dose is up to 40 mg, up to 35 mg, 30-35 mg, or up to 30 mg per kg of bodyweight of a subject. Using DHC in low doses is attractive because it minimises the risk of side effects such as bile stones.

It may be particularly beneficial if the DHC, in the low doses described herein, is used in conjunction with a diet and/or exercise program because this may positively affect the ratio between low density lipoprotein (hereinafter LDL) and high density lipoprotein (hereinafter HDL) transported lipids e.g. cholesterol and/or triglycerides.

The use of DHC as described herein may be particularly relevant for elderly subjects because this group is more likely to suffer from hyperlipidemia.

The DHC may be administered enterally e.g. orally to a subject e.g. a mammal in any form e.g. in its pure form or in the form of a composition e.g. in the form of a nutritional product, a food product, a functional food product, a healthy ageing product, a dairy product, a nutritional supplement, a pharmaceutical formulation, a beverage product, a diet, or a pet food product. Such compositions are provided herein and may comprise DHC in any amount, but ordinarily will contain DHC within a concentration range selected from the group consisting of: 0.01-0.4%, 0.02-0.38%, 0.05-0.36%, 0.1-0.34%, 0.15-0.32%, or 0.2-0.3% by weight of the composition.

DHC or compositions comprising DHC may be included in a kit further comprising a label indicating dosage requirements that correspond/equate to a dosage of DHC as defined herein.

### DESCRIPTION OF THE DRAWINGS

FIG. 1a represents the chemical structure of dihydrocholesterol (DHC).

FIG. 1b represents the chemical structure of cholesterol.

FIG. 2 represents the study design of the hyperlipidemia hamster model used for analyzing the effects of dihydrocholesterol (DHC) on plasma cholesterol (Example 6).

FIG. 3 represents changes in plasma total cholesterol (TC), non-HDL cholesterol (nHDL-C), and total triacylglycerides (TG) in week 6, in hamsters fed with the non-cholesterol diet (NCD), high cholesterol diet (HCD) and four experimental diets supplemented with 0.2% DHC (DA), 0.3% DHC (DB), 0.2% β-sitosterol (SA) and 0.3% β-sitosterol (SB) (Example 6). Data were expressed as mean±SD; n=7 for NCD, HCD and DB, n=8 for DA, SA and SB; means at the same row with different superscript (a, b, c, d) differ significantly at p<0.05.

FIG. 4 represents fecal excretion of neutral and acidic sterols in week 1 and 6 in hamsters fed with the non-cholesterol diet (NCD), high cholesterol diet (HCD) and four experimental diets supplemented with 0.2% DHC (DA), 0.3% DHC (DB), 0.2% β-sitosterol (SA) and 0.3% β-sitosterol (SB) (Example 6). Data were expressed as mean±SD; n=7 for NCD, HCD and DB, n=8 for DA, SA and SB; means at the same row with different superscript (a, b, c, d, e) differ significantly at p<0.05

FIG. 5 represents the effect of dietary dihydrocholesterol (DHC) and β-sitosterol on (a) atherosclerotic plague and (b) liver cholesterol and dihydrocholesterol in hamsters fed with the non-cholesterol diet (NCD), high cholesterol diet (HCD) and four experimental diets supplemented with 0.2% DHC (DA), 0.3% DHC (DB), 0.2% β-sitosterol (SA) and 0.3% β-sitosterol (SB) (Example 6). Values were expressed as means±SD (n=7 for NCD, HCD and DB, n=8 for DA, SA, and SB). Means with different superscript letters differ significantly, p<0.05.

FIG. 6 represents the effect of dietary dihydrocholesterol (DHC) and sitosterol on protein mass levels and mRNA levels of LDL receptor, liver X receptor alpha (LXRα), and cholesterol-7α-hydroxylase (CYP7A1) in hamsters fed with the non-cholesterol diet (NCD), high cholesterol diet (HCD) and four experimental diets supplemented with 0.2% DHC (DA), 0.3% DHC (DB), 0.2% β-sitosterol (SA) and 0.3% β-sitosterol (SB) (Example 6). For the protein mass levels, data were normalized with β-actin. For the mRNA levels, data are normalized with GAPDH. Values were expressed as means±SD (n=7 for NCD, HCD and DB, n=8 for DA, SA, and SB) with those for the negative control group being arbitrarily taken as one. Means with different superscript letters differ significantly, p<0.05.

FIG. 7 represents the effect of dietary dihydrocholesterol (DHC) and β-sitosterol on mRNA levels of intestinal Niemann-Pick C1 like 1 (NPC1L1), acyl coenzyme A: cholesterol acyltransferase 2 (ACAT2), ATP binding cassette transporters (ABCG5 and ABCG8) in hamsters fed with the non-cholesterol diet (NCD), high cholesterol diet (HCD) and four experimental diets supplemented with 0.2% DHC (DA), 0.3% DHC (DB), 0.2% β-sitosterol (SA) and 0.3% β-sitosterol (SB) (Example 6). Data are normalized with cyclophilin. Values were expressed as means±SD (n=7 for NCD, HCD and DB, n=8 for DA, SA, and SB) with those for the negative control group being arbitrarily taken as one. Means with different superscript letters differ significantly, p<0.05.

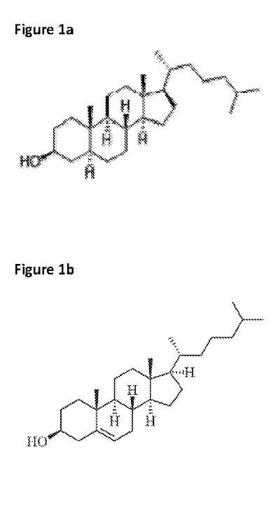

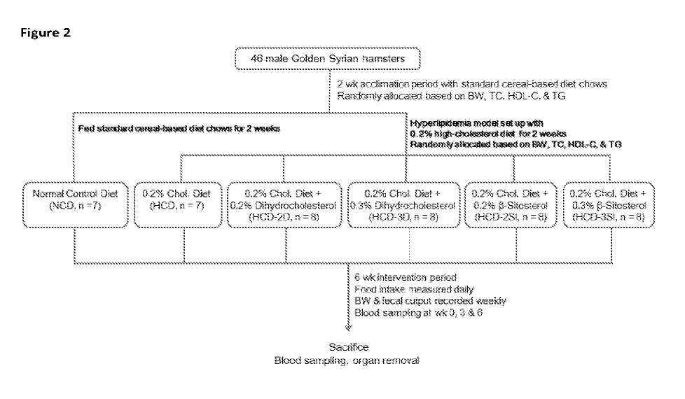

### DETAILED DESCRIPTION

As stated herein above, it has surprisingly been found that a low dose of DHC of up to 50 mg per kg of body weight has an effect in minimising weight gain and/or lowering plasma lipid levels, in particular plasma cholesterol levels and plasma triglyceride levels.

In a first aspect of the present invention there is provided DHC in a dose of up to 50 mg per kg of body weight to treat and/or prevent excess weight or obesity and/or hyperlipidemia, and/or a disorder associated with any of the foregoing.

In another aspect there is provided DHC for use in the manufacture of a medicament for use to treat and/or prevent excess weight or obesity and/or hyperlipidemia, and/or a disorder associated with any of the foregoing, wherein said medicament is administered in a dose equating or corresponding to up to 50 mg of DHC per kg of body weight.

In another aspect there is provided a method to treat and/or prevent excess weight or obesity and/or hyperlipidemia, and/or a disorder associated with any of the foregoing, comprising administering DHC in a dose of up to 50 mg per kg of body weight.

Advantageously, when DHC was used in a low dose as specified herein, side effects were absent or minimised. Accordingly, when used at a dose specified herein, DHC can not only exert beneficial effects on weight and plasma lipid levels, more particularly plasma cholesterol levels and plasma triglyceride levels, it can do this whilst minimising the risk of or avoiding potential side effects e.g. bile stones.

The term “body weight” as used herein refers to a subjects mass or weight.

The term “dose” as used herein refers to a daily quantity of DHC that is administered to a subject.

The daily quantity or dose of DHC may be administered all at once or it may be spread out over several administrations throughout a day.

The term “subject” as used herein refers to a mammal and more particularly a cat, a dog or a human.

The DHC in the doses specified herein can be administered to a subject by any known method. In particular the DHC can be administered enterally e.g. orally.

Particular useful doses of DHC may be up to 40 mg, up to 35 mg, 30 to 35 mg, or up to 30 mg per kg of bodyweight.

In an embodiment of the invention, DHC in the doses specified herein, is used in a human with a BMI of over 25, more particularly over 30.

As stated herein above, a human is considered as having excess weight if they have a BMI of more than 20. If a human has a BMI of more than 30 they are considered to be obese.

The term “hyperlipidemia” as used herein refers to any abnormally elevated level of any or all lipids and/or lipoproteins in the plasma and includes hypercholesterolemia, hypertriglyceridemia and a combination thereof.

In the context of the present invention hyperlipidemia may be primary hyperlipidemia which is usually due to genetic causes, or secondary (acquired) hyperlipidemia resulting from another underlying disorder that leads to alterations in plasma lipid and lipoprotein metabolism such as diabetes mellitus type II, hypertension, central obesity and insulin resistance (“syndrome X”). Hyperlipidemia may also be of the idiopathic type, where the cause is unknown.

The term “hypertriglyceridemia” as used herein refers to abnormally elevated levels of triglycerides in the plasma. The US national institute of health classifies a total triglyceride level of less than 150 mg/dL in humans as desirable or good.

The term “hypercholesterolemia” as used herein refers to abnormally elevated levels of cholesterol in the plasma. The US National Institute of Health classifies total cholesterol of less than 200 mg/dL in humans as desirable or good.

In an embodiment of the invention, DHC in the doses specified herein, is used in a human with a total cholesterol level of above 200 mg/dL and/or a total triglyceride level of above 150 mg/dL.

Lipids, such as cholesterol and triglycerides, are transported in plasma in Lipoprotein molecules. Two specific types of lipoprotein molecules are low density lipoprotein (hereinafter LDL) molecules and high density lipoprotein (hereinafter HDL) molecules. Studies have shown that there is a positive correlation between the levels of lipids, e.g. cholesterol and triglycerides, transported in LDL molecules and a variety of health problems such as CVDs. In line with this the American Heart Association, and the National institute of Health (NIH), have provided a set of guidelines for fasting levels of cholesterol transported in LDL molecules (hereinafter LDL-Cholesterol) and risk for heart disease.

A fasting LDL-Cholesterol level of 100 to 129 mg/dL corresponds to a near optimal LDL level, corresponding to higher rates for developing symptomatic cardiovascular disease events.

A fasting LDL-Cholesterol level of 130 to 159 mg/dL corresponds to a borderline high LDL level, corresponding to higher rates for developing symptomatic cardiovascular disease events.

A fasting LDL-Cholesterol level of 160 to 199 mg/dL corresponds to a high LDL level, corresponding to much higher rates for developing symptomatic cardiovascular disease events.

Finally, a fasting LDL-Cholesterol level of above 200 mg/dL corresponds to a very high LDL level, corresponding to highest increased rates of symptomatic cardiovascular disease events

In an embodiment of the present invention DHC in the doses specified herein is used in a human with a fasting LDL cholesterol level of above 100 mg/dL, of above 130 mg/dL, of above 160 mg/dL or of above 200 mg/dL.

In contradistinction to LDL-lipids, higher concentrations of HDL-lipids correlate with a reduction in the risk of many diseases e.g. CVDs such as atherosclerosis. HDL molecules collect lipids e.g. phospholipids, cholesterol, and/or triglycerides from the body's cells and/or tissues, and take it back to the liver. HDL molecules are sometimes referred to as “good” lipoprotein because of the correlation of higher concentrations with lower rates of diseases e.g. atherosclerosis progression and/or regression. Advantageously, weight loss and/or exercise has been shown to increase HDL levels.

Accordingly, DHC may be advantageously used as defined herein in conjunction with a diet and/or exercise program i.e. calorie restricted regimen, cholesterol and/or triglyceride restricted regimen, and/or regular aerobic exercise. This may positively affect the LDL to HDL ratio.

DHC in the doses specified herein may be administered briefly before, with, or briefly after the consumption of food high in total lipids (high fat foods) or high in one or more specific lipid. This may prevent or minimise the absorption of one or more lipid comprised in said food e.g. cholesterol and/or triglycerides, in the gastrointestinal tract.

This may be particular advantageous in a mammal trying to maintain a constant and/or optimum plasma lipid level e.g. a constant and/or optimum cholesterol and/or triglyceride level, and may thereby prevent hyperlipidemia e.g. hypercholesterolemia and/or hypertriglyceridemia.

Food shall be considered high in lipids if it contains more than 25%, more than 20% or more than 17% fat.

Food shall be considered high in one or more specific lipid if it contains more than 50%, more than 30% or more than 20% of the recommended daily intake for said lipid e.g. cholesterol.

Excess weight or obesity and/or hyperlipidemia, in particular hypercholesterolemia and hypertriglyceridemia, are common in the general population, and are an associated with a variety of disorders. Such disorders are well known to those skilled in the art (see for example Bhatnagar D. et al., BMJ 2008; 337: a993; and Poirier P, et al. (2006) Obesity and cardiovascular disease: pathophysiology, evaluation, and effect of weight loss: an update of the 1997 American Heart Association Scientific Statement on Obesity and Heart Disease from the Obesity Committee of the Council on Nutrition, Physical Activity, and Metabolism. Circulation. 113: 898: 918, both of which are herewith incorporated by reference).

Excess weight or obesity and/or hyperlipidemia e.g. particular hypercholesterolemia and hypertriglyceridemia, may be associated with a disorder because it increases the risk of the development of that disorder.

Non limiting examples of disorders associated with hyperlipidemia, in particular hypercholesterolemia and/or hypertriglyceridemia, include: lipoprotein dysregulation, hyperlipidemia related cardio-cerebro-vascular diseases including coronary heart disease, angina, myocardial infarction, atherosclerosis, coronary artery disease, stroke, claudication, peripheral vascular disease, non-alcohol fatty liver disease, and combinations thereof.

Non limiting examples of disorders associated with excess weight or obesity include: metabolic diseases such as type II diabetes, CVDs such as coronary heart disease, heart failure, and sudden death because of the impact on the cardiovascular system

Since disorders associated with hyperlipidemia typically occur with ageing, the invention may be particularly relevant for adult or the elderly subjects.

For the purpose of the present invention a subject e.g. a human shall be considered as “elderly” if it has surpassed the first half of its average expected lifespan in its country of origin or for its species, preferably, if it has surpassed the first two thirds of the average expected lifespan in its country of origin or for its species, more preferably if it has surpassed the first three quarters of the average expected lifespan in its country of origin or for its species, most preferred if it has surpassed the first four fifths of the average expected lifespan in its country of origin or for its species. For humans this may for example be above the age of 45, 50, 55, 60, 65, 70, 75 or 80 years of age.

DHC in the doses specified herein may be used in any form, for example it may be used in its pure form or substantially pure form e.g. 80% to 99%, or 90% to 95% purity, or in the form of a composition (liquid or solid) that is suitable for consumption by a subject.

In another aspect of the present invention there is provided a composition comprising DHC.

Said composition may comprise up to 99.9% DHC by weight of the composition.

As will be evident to the skilled person the quantity of DHC comprised in a composition will depend on the nature of said composition. It is well within the purview of the skilled person to decide on the concentration of DHC to include in a composition depending on the nature of the composition and any further ingredients that may be comprised therein.

Non limiting examples of concentration ranges within which DHC can be included in the composition of the invention include; 0.01-0.4%, 0.02-0.38%, 0.05-0.36%, 0.1-0.34%, 0.15-0.32%, and 0.2-0.3% by weight of the composition.

The compositions of the invention may comprise any type of further ingredient that is suitable for consumption by a subject. Non limiting examples of further ingredients include nutrients, for instance, selected from the group of lipids (not comprising DHC), carbohydrates, and protein, micronutrients, or pharmaceutically active agents; conventional food additives such as anti-oxidants, stabilizers, emulsifiers, acidulants, thickeners, buffers or agents for pH adjustment, chelating agents, colorants, excipients, flavor agents, osmotic agents, pharmaceutically acceptable carriers, preservatives, sugars, sweeteners, texturizers, emulsifiers, water and any combination thereof.

Other suitable ingredients for consumable compositions are described in standard texts, such as “Handbook of Industrial Chemical Additives”, ed. M. and I. Ash, 2 nd Ed., (Synapse 2000).

In a particular embodiment the composition comprises cholesterol. Cholesterol can be comprised in the composition in any amount. Non limiting examples of concentration ranges for cholesterol include, 0.01 to-1%, 0.1-0.8%, 0.15 to 0.2%, and 0.2% by weight of the final composition.

The composition of the present invention may be any type of composition for example the composition may be a nutritional product, a food product, a functional food product, a healthy ageing product, a dairy product, a nutritional supplement, a pharmaceutical formulation, a beverage product, a diet, or a pet food product.

The term “food product”, as used herein, refers to any kind of product that may be safely consumed by a subject e.g. a human or an animal. Said food product may be in solid, semi-solid or liquid form and may comprise one or more nutrients, foods or nutritional supplements. For instance, the food product may additional comprise the following nutrients and micronutrients: a source of proteins, a source of lipids, a source of carbohydrates, vitamins and minerals. The composition may also contain anti-oxidants, stabilizers (when provided in solid form) or emulsifiers (when provided in liquid form).

In the context of the present invention, the term “functional food product” is to be understood as a food product providing an additional health-promoting or disease-preventing function to a subject. An additional health-promoting function can be conferred to the individual by DHC comprised in the inventive composition, in terms of the total plasma lipid e.g. cholesterol and/or triglyceride reducing effect. Further known biologically-active compounds may be added to the food product of the invention in order to provide additional health benefits.

As used herein, a “healthy ageing product” can be a diet or nutritional supplement that is intended as a means to extend lifespan in a subject. Such a product may additionally contain antioxidants or other compounds such as dietary fiber, plant sterols, fish oils, MUFA, PUFA, flavones, polyphenols, lycopene, traditional Chinese ingredients such as hawthorn, kudzu, soybean, gingko, garlic, red yeast rice, walnuts, and combinations thereof.

As used herein, “antioxidants” are molecules capable of slowing or preventing the oxidation of other molecules. Preferably, antioxidants are selected from: beta-carotene, vitamin C, vitamin E, selenium, carotenoids, coenzyme Q10, flavonoids, glutathione, lutein, lycopene, polyphenols, vitamin A, vitamin B1, vitamin B6, vitamin B12, vitamin C, vitamin D, vitamin E, zeaxanthin, lipoic acid, carnosine, N-acetylcysteine, or combinations thereof.

The term “nutritional supplement”, or “dietary supplement”, as used herein, is to be understood as relating to a nutritional product that provides nutrients to a subject that may otherwise not be consumed in sufficient quantities by said individual. For instance, a nutritional supplement may include vitamins, minerals, fiber, fatty acids, or amino acids.

Dairy products, as used herein, are food products produced from animals such as cows, goats, sheep, yaks, horses, camels, and other mammals. Examples of dairy products suitable in the present invention are low-fat milk (e.g. 0.1%, 0.5% or 1.5% fat), fat-free milk, milk powder, whole milk, whole milk products, butter, buttermilk, buttermilk products, skim milk, skim milk products, high milk-fat products, condensed milk, créme fraiche, cheese, ice cream and confectionery products. Preferably, the dairy product is selected from a low-fat milk, a fat-free milk, a milk product, or a protein powder.

A pharmaceutical formulation, as used herein, is to be understood as comprising at least one pharmaceutically active agent, chemical substance or drug. The pharmaceutical formulation may be in solid or liquid form and can comprise at least one additional active agent, carrier, vehicle, excipient, or auxiliary agent identifiable by a person skilled in the art. The pharmaceutical formulation can be in the form of a tablet, capsule, granules, powder, liquid or sirup. The pharmaceutically active agent, chemical substance or drug may be dihydrocholesterol (DHC) itself or be selected from one or more additional active agents useful for treating dyslipidemia and cardiovascular disease. For instance, said additional active agents may be selected from the group consisting of HMG-CoA reductase inhibitors (statins), fibrates, nicotinic acid, cholestyramine, etc.

A beverage product is a nutritional product in liquid or semi-liquid form that may be safely consumed by a subject.

A pet food product is a nutritional product that is intended for consumption by pets e.g. dogs, cats, rodents such as mice, rats, and guinea pigs, rabbits, etc.

In an embodiment the composition is a low-fat milk, a fat-free milk, a milk product, or a protein powder.

In another aspect of the present invention there is provided a method for producing the above described composition comprising the steps of a) providing DHC, (b) providing at least one further ingredient, (c) mixing DHC and said at least one further ingredient, (d) thereby obtaining said composition.

DHC is freely available from many supplier including Sigma-Aldrich.

The present method may optionally comprise a further step of packaging the composition in a suitable container such as a flask, box, jar, blister, etc.

In another aspect of the present invention there is provided a kit comprising at least two individual parts of the final composition of the invention. The parts of the kit can be mixed to yield the final composition.

One part in the kit can provide DHC or a composition comprising dihydrocholesterol (DHC).

The remaining part(s) of the kit can provide at least one further ingredient to be mixed with said DHC or said composition comprising DHC.

In another aspect of the present invention there is provided a kit for providing a low dose of DHC up to 50 mg per kg of body weight, the kit comprising:
a) DHC or a composition comprising DHC as defined herein
b) A label indicating dosage requirements for said DHC or composition equating or corresponding to a dosage of DHC of up to 50 mg per kg of body weight as specified herein.

The dosage requirements may be with respect to the quantity of said composition and/or the consumption frequency e.g. the number of times or servings per day.

It will be evident to the skilled person that the amount of DHC, or a composition comprising DHC, that will need to be administered to a subject will depend on the body weight of said subject, and in the case of a composition comprising DHC, on the concentration of DHC comprised within said composition.

Those skilled in the art will understand that they can freely combine all features of the present invention disclosed herein. In particular, features described for different embodiments of the present invention may be combined. Further advantages and features of the present invention are apparent from the figures and examples.

### EXAMPLES

### Example 1: Composition of Dairy Product Containing DHC

| Dairy Base | Composition (%) |
|---|---|
| Full Cream Milk Powder | 56.0 |
| Corn Syrup | 16.1 |
| Whey Sweet | 11.0 |
| Oil Mix | 7.0 |
| Lactose Monohydrate | 3.3 |
| Trace Element Premix | 2.0 |
| Vitamin Premix DAIRY | 0.3 |
| Flavor Milk | 0.6 |
| DHC | 0.2 |
| Water | 3.5 |

### Example 2: Composition of Protein Powder Containing DHC

| Protein Powder Base | Composition (%) |
|---|---|
| Protein Whey Powder | 5.0 |
| Soya Protein Powder | 52.0 |
| Sugar Powder | 15.0 |
| Maltodextrin | 23.4 |
| Xanthan | 0.3 |
| Probiotic Preblend PP047 | 0.2 |
| Flavor | 0.4 |
| DHC | 0.2 |
| Water | 3.5 |

### Example 3: Composition of Pet Food Products Containing DHC

| Pet Food Base (Especially for hamsters) | Composition (%) |
|---|---|
| Corn starch | 508 |
| Casein | 242 |
| Sucrose | 119 |
| Lard | 50 |
| Mineral mixture AIN-76 | 40 |
| Vitamin mixture AIN-76A | 20 |
| DL-methionine | 1 |
| Dihydrocholesterol | 2 |

### Example 4—Analysis of the Effects of Dihydrocholesterol (DHC) on Blood Lipid Reduction by Means of a Hamster Hyperlipidemia Model

### Experimental Diets

Six different experimental diets were prepared by mixing the ingredients according to Table 1 and blending these mixtures with a gelatin solution (20 g/L) in a ratio of 200 g composition per liter of solution. Once the gelatin had set, the product were cut into pieces of approximately 10 g cubes and stored frozen at −80° C. until use.

TABLE 1

| Ingredients (g/kg diet) | NCD | HCD | HCD-2D | HCD-3D | HCD-2SI | HCD-3SI |
|---|---|---|---|---|---|---|
| Cholesterol | — | 2 | 2 | 2 | 2 | 2 |
| Dihydrocholesterol | — | — | 2 | 3 | — | — |
| B-Sitosterol | — | — | — | — | 2 | 3 |
| DL-methionine | 1 | 1 | 1 | 1 | 1 | 1 |
| Corn starch | 508 | 508 | 508 | 508 | 508 | 508 |
| Casein | 242 | 242 | 242 | 242 | 242 | 242 |
| Lard | 50 | 50 | 50 | 50 | 50 | 50 |
| Sucrose | 119 | 119 | 119 | 119 | 119 | 119 |
| Mineral mixture AIN-76 | 40 | 40 | 40 | 40 | 40 | 40 |
| Vitamin mixture AIN-76A | 20 | 20 | 20 | 20 | 20 | 20 |

NCD is a non-cholesterol diet comprising corn starch, casein, sucrose, lard, mineral mix, vitamin mix, and DL-methionine in the amounts specified in Table 1.

HCD is a high-cholesterol diet that was prepared by adding 0.2% (w/w) cholesterol into NCD.

DA and DB were prepared by supplementing the HCD diet with 0.2% DHC and 0.3% DHC, respectively.

SA and SB were prepared by supplementing the HCD diet with 0.2% β-sitosterol and 0.3% β-sitosterol, respectively.

### Animal Design

46 male Golden Syrian hamsters (n=46; body weights=110-120 g) were housed in wire-bottomed cages at 23° C. in an animal room with 12-hour light-dark cycle (one per cage). All hamsters were maintained on a standard cereal based diet for two weeks.

After this acclimation period the hamsters were randomly divided into six test groups and fed one of the experimental diets for another six weeks.

The test groups were the following:

NCD: Normal control diet (No cholesterol added) group fed standard cereal-based diet chows (n=7)
HCD: High (0.2%) Cholesterol Diet group (n=7)
DA: High (0.2%) Cholesterol+0.2% Dihydrocholesterol Diet (HCD-2D) group (n=8)
DB: High (0.2%) Cholesterol+0.3% Dihydrocholesterol Diet (HCD-3D) group (n=8)
SA: High (0.2%) Cholesterol+0.2% β-Sitosterol Diet (HCD-2SI) group (n=8)
SB: High (0.2%) Cholesterol+0.3% β-Sitosterol Diet (HCD-3SI) group (n=8)

Diets and water were given ad libitum and the food intake was measure daily. All hamsters were weighed and their total feces per cage were collected weekly. Blood sampling was performed at the beginning of week 1 and the end of week 3 and 6. Therefore, the hamsters were fasted overnight, and a 0.5 ml blood sample was obtained from the retro-orbital sinus into a heparinized capillary tube under inhalational anesthesia of isoflurane (100%). Following the last blood sample collection at week 6, all the hamsters were killed by carbon dioxide suffocation. The liver, heart, kidney, epididymal and perirenal adipose tissues and aorta were removed, washed in saline, and weighed. The first 10 cm of duodenum was discarded, and the next 30 cm of the small intestine was kept. All tissue samples were flash frozen in liquid nitrogen and stored at −80° C. until analysis.

### Analysis of Plasma Lipoproteins

Plasma total cholesterol (TC) and triacylglycerols (TG) were quantified using commercial enzymatic kits from Infinity (Waltham, Mass., U.S.A) and Stanbio Laboratories (Boerne, Tex., U.S.A.), respectively. To determine high-density lipoprotein cholesterol (HDL), low-density lipoprotein cholesterol (LDL) and very low-density lipoprotein cholesterol (VLDL) were first precipitated with a commercial kit (Stanbio) containing phosphotungstic acid and magnesium chloride. HDL cholesterol in supernatant phase was measured similarly as done for TC. Non-HDL cholesterol was calculated by deducing HDL cholesterol from TC.

### Analysis of Atherosclerotic Plaques

The percentage area of atherosclerotic plaque on endothelial layer was determined. The thoracic aorta was cut opened vertically. The aortas were stained with 1 ml saturated oil red in isopropanol before being scanned with a table scanner (Epson 1220 perfection, Epson Co., Japan). The area of atherosclerotic plaque was measured by means of a computer image analyzing program “Sigma Scan Pro 5.0” (SPSS, Inc., Chicago, USA).

### Analysis of Cholesterol and its Derivatives in Organs

Cholesterol content in organs was determined by a standard method. Cholesterol in the tissue sample was calculated according to the amount of internal standard 5α-cholestanol added.

### Analysis of Fecal Neutral and Acidic Sterols

Neutral and acidic sterols in the feces were quantified by a standard method using gas chromatography (GC). In brief, the total fecal sample (300 mg) from each hamster was freeze-dried, ground and well mixed. 0.3 mg of 5α-cholestanol was added as an internal standard for quantification of neutral sterols by GC analysis. The remaining aqueous layer was saved for the analysis of acidic sterols.

### Results

### Food Intake, Body and Organ Weight

No differences in food intake, initial and final body weights were seen among the six groups (see Table 2). When organ weights were expressed as gram per 100 gram body weight, no differences in weights of heart, testis and peri-renal fat pad were seen among the six groups. However, groups DA, DB, SA and SB had a decreased liver weight compared with the HCD group. DB and SA groups had a reduced kidney weight compared with HCD, while the DA and SB group showed no significant difference. DA, SA and SB hamster had a decreased epididymal fat pad weight compared with the control (see Table 2). An excess of visceral fat is known as central obesity, which has a strong correlation with cardiovascular disease. Visceral fat is composed of several adipose depots including mesenteric fat, epididymal white adipose tissue (EWAT) and perirenal fat. In such case, decreased epididymal fat in the current study could be regarded as an indicator that DHC supplementation might be effective to prevent/treat excess weight or obesity, and subsequently have benefit for cardiovascular health (see Yusuf S, Hawken S, Ounpuu S, Dans T, Avezum A, Lanas F, McQueen M, Budaj A, Pais P, Varigos J, Lisheng L, INTERHEART Study Investigators. (2004). “Effect of potentially modifiable risk factors associated with myocardial infarction in 52 countries (the INTERHEART study): case-control study”. Lancet 364 (9438): 937-52).

### Plasma TC, HDL-C, LDL/HDL, HDL/TC and TG

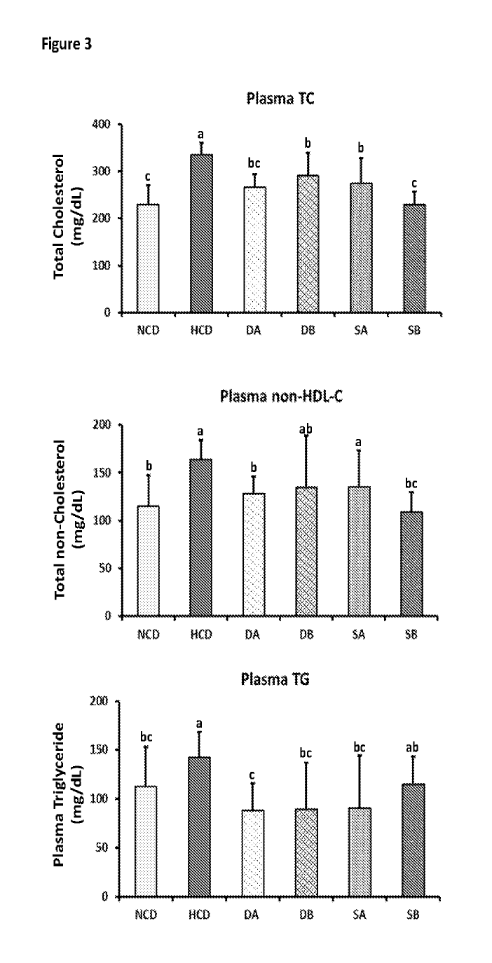

All experimental groups of hamsters had similar levels of plasma TC, non-HDL-C and TG at week 0. When the experiment reached the end of week 6, DA, DB, SA and SB hamsters had plasma TC, non-HDL-C and TG significantly lower than the HCD group. β-sitosterol at 0.3% was more effective than DHC in reducing plasma TC (see FIG. 3).

### Fecal Total Sterols

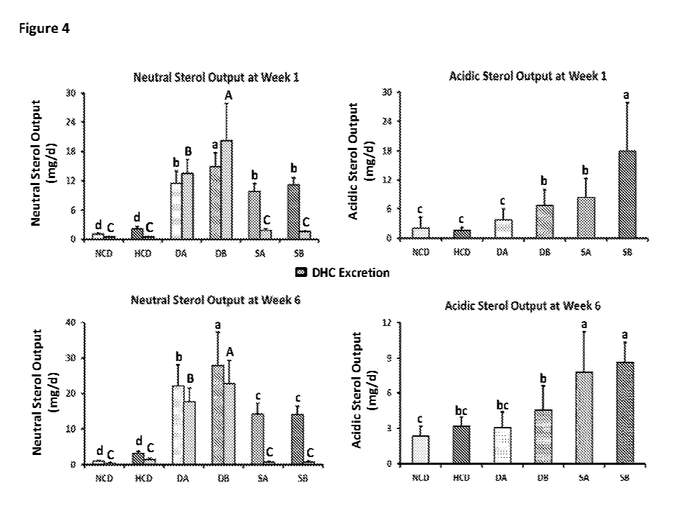

At week 6, DA, DB, SA and SB hamsters excreted total fecal neutral sterols greater than the controls. DA and DB groups showed greater excretion of total neutral sterols than their corresponding SA and SB groups. In contrast, SA and SB groups excreted greater amount of acidic sterols than their corresponding DA and DB groups (see FIG. 4).

### Atherosclerotic Plague

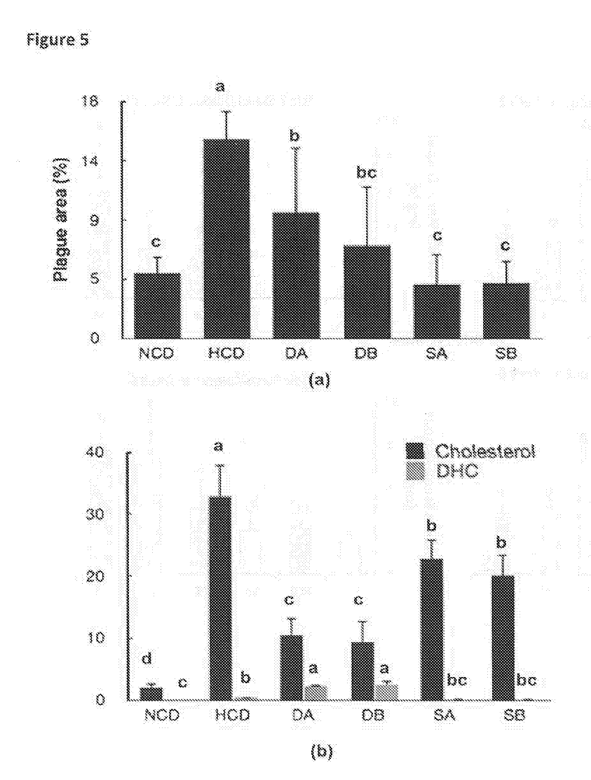

DA, DB, SA and SB hamsters had atherosclerotic plague significantly lesser than the HCD control group (FIG. 5a). SA and SB diets were more effective than DA and DB diets in reducing the formation of atherosclerotic plague (FIG. 5a).

### Liver Cholesterol and Dihydrocholesterol

DA, DB, SA and SB hamsters had hepatic cholesterol levels significantly lower than the HCD control (FIG. 5b). DA and DB diets were more effective than SA and SB in reducing the liver cholesterol (FIG. 5b). DA and DB hamsters accumulated about 2 mg DHC/g liver while SA and SB accumulated very little DHC (0.2 mg/g) in the liver.

TABLE 2
Changes in food intake, body weight, relative organ weights (100 g total body weight) in hamsters fed with the
non-cholesterol diet (NCD), high cholesterol diet (HCD) and four experimental diets supplemented with 0.2%
DHC (DA), 0.3% DHC (DB), 0.2% β-sitosterol (SA) and 0.3% β-sitosterol (SB).

| | NCD | HCD | DA | DB | SA | SB |
|---|---|---|---|---|---|---|
| Daily Food Intake | 10.99 ± 0.77 | 10.55 ± 0.57 | 9.79 ± 0.67 | 10.30 ± 1.02 | 10.71 ± 1.20 | 10.56 ± 0.79 |
| (g/hamster) | | | | | | |
| Body Weight (g) | | | | | | |
| Initial | 107.71 ± 7.57 | 108.14 ± 9.27 | 101.25 ± 5.80 | 104.00 ± 8.23 | 103.88 ± 5.00 | 104.38 ± 6.61 |
| Final | 116.43 ± 7.76 | 120.43 ± 11.93 | 115.38 ± 9.02 | 115.43 ± 7.00 | 118.43 ± 13.74 | 112.00 ± 8.05 |
| Relative Organ weight | | | | | | |
| (% Body Weight) | | | | | | |
| Liver | 3.88 ± 0.66^{bc} | 5.36 ± 0.28^{a} | 4.30 ± 0.28^{b} | 4.63 ± 0.28^{b} | 4.36 ± 0.70^{b} | 4.20 ± 0.48^{b} |
| Heart | 0.34 ± 0.05 | 0.48 ± 0.12 | 0.37 ± 0.09 | 0.42 ± 0.20 | 0.33 ± 0.11 | 0.42 ± 0.06 |
| Kidney | 1.13 ± 0.06^{ab} | 1.21 ± 0.14^{a} | 1.06 ± 0.08^{ab} | 1.02 ± 0.25^{b} | 0.99 ± 0.10^{b} | 1.17 ± 0.09^{a} |
| Testis | 1.54 ± 1.15 | 2.37 ± 1.08 | 2.12 ± 1.19 | 2.70 ± 0.64 | 2.24 ± 1.28 | 2.53 ± 0.87 |
| Epididymal | 1.34 ± 0.14^{b} | 1.76 ± 0.32^{a} | 1.30 ± 0.21^{b} | 1.55 ± 0.42^{ab} | 1.32 ± 0.31^{b} | 1.42 ± 0.29^{b} |
| Perirenal | 0.89 ± 0.14 | 1.03 ± 0.19 | 0.93 ± 0.31 | 1.01 ± 0.33 | 0.76 ± 0.25 | 0.89 ± 0.24 |

### Immunoblot of Hepatic SREBP-2, LDL Receptor, HMGR, LXRα and CYP7A1

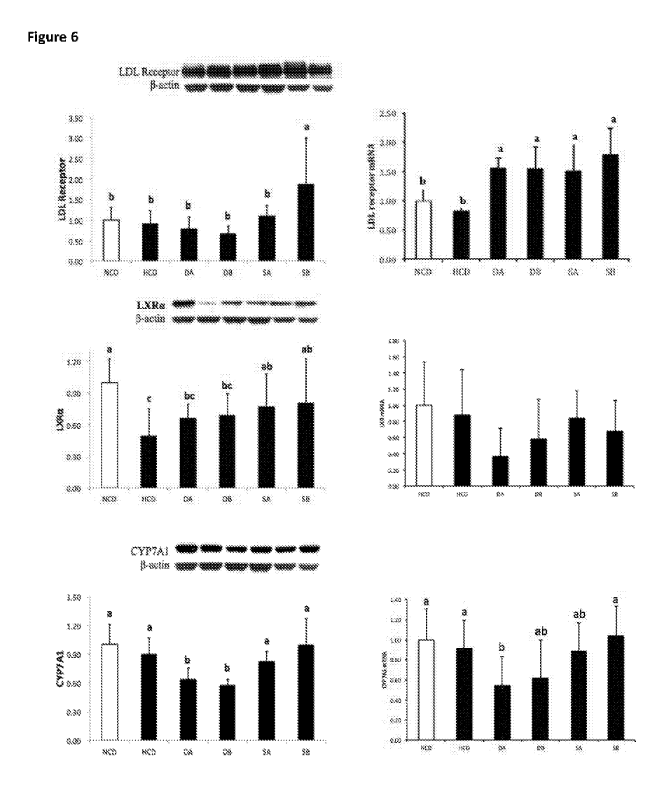

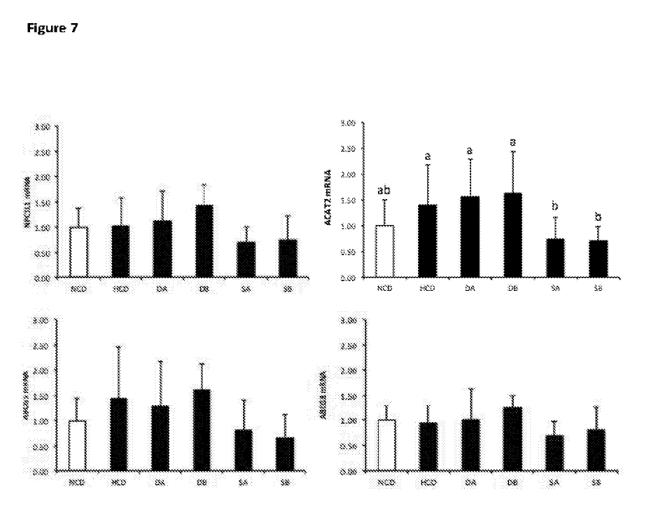

The western blot analysis demonstrated that both DA and DB diets but not SA and SB diets were able to down-regulate the protein mass of CYP7A1 compared with the HCD diet (FIG. 6, left). The SB diet significantly up-regulated the LDL receptor protein. DA and DB diets up-regulated LXRα slightly, while SA and SB significantly up-regulated LXRα (FIG. 6, left).

mRNA of Hepatic SREBP-2, HMGR, LDL Receptor, LXRα and CYP7A1

DA, DB, SA and SB hamsters up-regulated the mRNA level of the LDL receptor compared with the HCD control (FIG. 6, right). The DA diet down-regulated mRNA CYP7A1 compared with the DB, SA and SB diets.

### CONCLUSION

Example 6 clearly demonstrates that dietary DHC in a concentration of 0.2 and 0.3% by weight of the composition, respectively, significantly reduces plasma total cholesterol (TC) and triacylglycerol (TG) levels. Both, DHC and β-sitosterol, were shown to decrease plasma HDL and non-HDL cholesterol without affecting their ratio. However, the effect of DHC differed from that of β-sitosterol. First, the cholesterol-lowering effect of DHC was not dose-dependent. In contrast, β-sitosterol had a similar cholesterol-lowering activity but the effect was dose-dependent. Second, DHC was demonstrated to be more effective than β-sitosterol in reducing serum TG. The cholesterol-lowering activity of DHC may be effected by two potential mechanisms. First, DHC may stimulate the excretion of fecal neutral sterols and inhibit cholesterol absorption. This was evidenced in that excretion of cholesterol and its microbial derivatives increased 11.5-fold and 19.1-fold in DA and DB groups, respectively, compared with the HCD control. Second, there was a greater decrease in liver cholesterol by 67.5% and 71.5% in DA and DB groups compared with that in the corresponding groups, SA and SB (30.5% and 38.4%). The reduction in liver cholesterol may cause up-regulation of hepatic mRNA LDL receptor, leading to reduction in serum cholesterol concentration.

In conclusion, it was demonstrated that DHC in the claimed concentrations exhibits a strong cholesterol and triglycerides lowering activity and, thus, represents a suitable agent in the treatment of hyperlipidemia in an individual. DHC was found to be effective in reducing the formation of atherosclerotic plagues, inhibiting cholesterol absorption, stimulating the excretion of fecal neutral sterols, reducing hepatic cholesterol levels and stimulating the up-regulation of hepatic mRNA LDL receptors.

1. A method for treating and/or preventing excess weight or obesity and/or hyperlipidemia, and/or a disorder associated with any of the foregoing comprising administering dihydrocholesterol at a dose of up to 50 mg per kg of body weight to an individual in need of same.

2. Method according to claim 1 wherein, the dihydrocholesterol is used at a dose of up to 40 mg per kg of body weight.

3. Method according to claim 1 wherein, the disorder associated with excess weight or obesity and/or hyperlipidemia is selected from the group consisting of; lipoprotein dysregulation, hyperlipidemia related cardio-cerebro-vascular diseases including coronary heart disease, angina, myocardial infarction, atherosclerosis, coronary artery disease, stroke, claudication, peripheral vascular disease, metabolic diseases such as obesity, fatty liver disease, type II diabetes and combinations thereof.

4. Method according to claim 1 wherein hyperlipidemia is selected from the group consisting of hypercholesterolemia, hypertriglyceridemia, and combinations thereof.

5. Method according to claim 1 wherein the individual is selected from the group consisting of: human, cat, and dog.

6. Method according to claim 1 wherein the DHC is administered enterally.

7. Method according to claim 1 wherein the DHC is used in conjunction with a diet and/or exercise program.

8. Method according to claim 5 wherein the individual is elderly.

9. Method according to claim 1 wherein said DHC is used in the form of a composition selected from the group consisting of: a nutritional product, a food product, a functional food product, a healthy ageing product, a dairy product, a nutritional supplement, a pharmaceutical formulation, a beverage product, a diet, and a pet food product.

10. A composition comprising dihydrocholesterol at a concentration of 0.01 to 0.4% by weight.

11. A composition according to claim 10 selected from the group consisting of: a nutritional product, a food product, a functional food product, a healthy ageing product, a dairy product, a nutritional supplement, a pharmaceutical formulation, a beverage product, a diet, and a pet food product.

12. A composition according to claim 11 wherein the compositions is a product selected from the group consisting of: a low-fat milk, a fat-free milk, a milk product, and a protein powder.

13-14. (canceled)

15. A kit for providing dihydrocholesterol in a low dose up to 50 mg per kg of body weight, the kit comprising:
DHC or a composition comprising DHC; and
a label indicating dosage requirements for said DHC, or the composition, so that the dose equates or corresponds to a dose of DHC of up to 50 mg per kg of body weight.
